(19) United States
(12) Patent Application Publication
(10) Pub. No.: US 2017/0042801 A1
(43) Pub. Date: Feb. 16, 2017

(54) SKIN CREAM COMPOSITIONS

(71) Applicant: Mario Medri, Miami Lakes, FL (US)

(72) Inventor: Mario Medri, Miami Lakes, FL (US)

(21) Appl. No.: 14/826,320

(22) Filed: Aug. 14, 2015

(51) Int. Cl. A61K 8/98; A61K 8/55; A61K 8/92; A61K 8/891; A61K 8/67; A61K 35/20; A61K 47/32; A61K 47/24; A61K 36/63; A61K 47/34; A61K 31/352; A61K 31/07; A61K 9/06; A61Q 19/00; A61Q 19/08; A61K 8/81

(52) U.S. Cl. CPC A61K 8/986; A61K 8/8158; A61K 8/553; A61K 8/922; A61K 8/891; A61K 8/678; A61K 8/671; A61K 35/20; A61K 47/32; A61K 47/24; A61K 36/63; A61K 47/34; A61K 31/352; A61K 31/07; A61K 9/06; A61Q 19/007; A61Q 19/00; A61Q 19/08; A61K 2800/30

(57) ABSTRACT

The invention provides a skin cream comprising: about 0.5 to 15% of colostrum; about 0.5 to 15% of a polyacrylamide based emulsifier; about 1.0 to 20% of an emulsifier containing lecithin or a derivative thereof; about 1.0 to 20% of olive oil, having an acidity ranging from about 0.2 to 2.0% and a usage range of 5.0% to 50% about 1.0 to 10% of dimethicone; about 0.01% to 4.0% of alpha-tocopherol or an active derivative thereof; about 0.01% to 0.5% of vitamin A or an active vitamin A derivative; about 0.5 to 4.0% of a natural preservative; and about 10 to 90% of purified water to make up 100 percent. Application of the skin cream of the present invention should smoothly lubricate and moisturize the user's skin, and lessen the appearance of wrinkles. The skin cream of the present invention will make the skin appear smoother and more vibrant.

### FIELD OF THE INVENTION

The present invention relates to all material skin cream compositions, and more particularly to all-natural skin cream compositions containing bovine colostrum to improve skin appearance and texture and improve or relieve various skin conditions including urticaria, dryness, eczema, and psoriasis,

### BACKGROUND OF THE INVENTION

The beauty market spurred by a desire to maintain youthful looks, even as one ages has given rise to numerous creams, treatments, and the like that seek to avoid or ameliorate formation of wrinkles. The aisles of drug stores, department stores, and even discount stores are lined with numerous products, each claiming to have a beneficial effect on the skin, or to bleach age spots, moisturize, condition, soften, or firm the skin, particularly the skin on women's faces and around their eyes, where signs of aging appear most accurate or noticeable. Many skin care products, however, are also being marketed to men.

Of course, numerous skin creams claim numerous benefits. For example, U.S. Pat. No. 5,658,580 (Mausner), discusses a skin cream that claims to minimize age spots and improve skin color, using a formula that includes sodium lactate, fatty acid esters of vitamins C and E, witch hazel, and horsetail extract. U.S. Pat. No. 7,557,094 (Wadstein), discusses skin creams featuring chitosan derivatives to treat atopic dermatitis, eczema, and to improve skin thickness and elasticity, U.S. Pat. No. 8,637,075 (Pedersen) provides a skin cream formulation comprising colostrum and a hydrocolloid, which are bioconjugated. These compositions also said to be helpful for treating various skin conditions.

Other references also discuss use of colostrum in skin care products. For example, WO 1994021225 (Wadstein) adds colostrum in a composition including a conjugate of a vitamin derivative and cylodextrum among other ingredients. CN 1098677 relates to a skin care product including colostrum, surfactants, including glycerol monostearate, and PEG 400 dioleate and water.

Each of the foregoing references is incorporated herein by reference in its entirety. There exists a need for an all-natural skin care cream that includes colostrum without including any artificial ingredients.

### SUMMARY OF THE INVENTION

The invention provides a skin cream comprising: about 0.5 to 15% of colostrum; about 0.5 to 15% of a polyacrylamide based emulsifier; about 1.0 to 20% of an emulsifier containing lecithin or a derivative thereof; about 1.0 to 20% of olive oil, having an acidity ranging from about 0.2 to 2.0% and a usage range of 5.0% to 50%; about 1.0 to 10% of dimethicone; about 0.01% to 4.0% of alpha-tocopherol or an active derivative thereof; about 0.01% to 0.5% of vitamin A or an active vitamin A derivative; about 0.5 to 4.0% of a natural preservative; and about 10 to 90% of purified water to make up 100 percent, The invention further provides a skin cream wherein the colostrum is bovine, ovine, porcine, seuine, or human derived, or a mixture thereof. The invention also provides a skin cream wherein the dimethicone is cetyl dimethicone. Application of the skin cream of the present invention should smoothly lubricate and moisturize the user's skin, and lessen the appearance of wrinkles. The skin cream of the present invention will make the skin appear smoother and more vibrant.

### DETAILED DESCRIPTION OF THE PREFERRED EMBODIMENTS

This invention relates to the preparation of a soft, smooth, easy to apply cosmetic cream for the enhancement and preservation of skin freshness and suppleness by the means of the addition of colostrum to the cream preparation. Such cream, being composed with all natural ingredients and to include specific amounts of ingredients such as vitamins, emulsifiers, antioxidant and others essential for the preservation of skin freshness, it is the ideal body treatment for the temporary preservation of epidermal freshness and subjective beauty. Colostrum, which is the first 48 hours post-partum milk, is incorporated in the various formulations of the creams and of specific compositions of the present invention depending of the intended targeted skin area to which it will be applied. The colostrum being used in these product preparations is of mammalian origin and in preferred form of bovine origin. Bovine colostrum is similar in its proteins, antibody and growth hormone and chemical composition to other mammals' colostrum, including that of humans. Bovine colostrum is the most convenient, abundant and commercially available type for this application. Colostrum has a GRAS status as it finds applications in food preparations for food enrichment. The present invention incorporates this ingredient into a topical class of cosmetic-like creams.

Bovine colostrum, also referred to as “colostrum” is the first milk secreted at the time of parturition, differing from the milk secreted later. It contains more lactalbumin and lactoprotein, and is also rich in antibodies that confer passive immunity to the newborn, it is sometimes called “foremilk.” Human Colostrum (HC) and Bovine Colostrum (BC) are both rich in protein, immunoglobulin, lactoferrin and growth factors. Recent studies suggest that colostrum components, immunoglobulin and growth factor benefit physically active persons and can be used to treat autoimmune and a wide variety of gastrointestinal conditions, including non-steroidal anti-inflammatory drug-induced gut injury, H. pylori infection, immune-deficiency related diarrhea, as well as infective diarrhea. See, http://www.foodandnutritionjournal.org/pdf/ vol1 no 1/1_1_4_p37_47_Colostrum_MEENA.pdf, and Current Research in Nutrition and Food Science, vol. 1(1), 37-47 (2013), both of which are incorporated by reference,

Humans have had a desire to preserve body freshness as an expression of reduced evidence of body aging. Throughout civilization, preservation of body freshness has been practiced to the extent that the Egyptian civilization extended the art of body preservation after physical death. Women were the first group of humans to practice body embellishment and primarily on the face and hands with the addition of botanical preparations to be applied to the skin, As time went on and knowledge increased, body beauty treatments became more and more sophisticated. Today, even in the most underdeveloped countries, we see the enhancement of feminine beauty by the means of cosmetics.

Today the beautification of the body, by the aid of creams, lotions, or perfumes (all identified as body treatments), has developed into a significant industry that includes both males and females. Cosmetic preparations have been and are being developed for the sole purpose of enhancing the illusion of beauty and the preservation of youthful appearance. Many creams are on the market today making a multitude of performance claims and consumers subjectively select the one that best fits their illusion of youth preservation.

The present invention relates to a cream that is primarily composed of all-natural ingredients. This cream has been formulated as a functional cream for different parts of the human body with the intent to help the epidermis to remain soft, supple and fresh against the challenge of time. The basic cream formula has been modified to be most effective for each different targeted area of the body. It is well known that the skin is basically the same throughout the body, but it is also known that it has different functional properties relating to specific part of the body. Skin elasticity is the most evident and apparent property that decreases in time and is at its best at a young age. Therefore, the current invention involves the addition of colostrum throughout the cream line independently of its specifically designed point of application. As we have seen, colostrum has significant valuable properties including the capability to restore sonic lost epidermal freshness through the absorption of specific proteins present in the colostrum, such as growth hormone, Igg, Iga, and Interferon, among others. The cream containing colostrum also delivers beneficial quantities of vitamins, emulsifiers and surfactants, all incorporated in a smooth, easy to apply, all natural formula.

Cream formulations are made to contain all natural ingredients and they are present in the following ranges. Bovine colostrum (Immuno Dynamics Inc.) in the range of about 0.5% to 15%; the emulsifier Sepigel EI 305 (Essential ingredients Inc.) in the range of about 1.0% to 20%; Lecithin (an emulsifier) (American Lecithin Inc.) in the range of about 0.1 to 5.0%; Olive Oil (Bertolli or any commercially available olive oil) with an acidity range from about 0.2% to 2.0% and in a usage range of about 5.0% to 50.0%; and Vitamin E as Alpha Tocopherol (HMS Sutton Labs) in the range of about 0.01% to 4.0%; Vitamin A as Retinol Palmitate or acetate (Glambia/BASF The Chemical Co.) in the range of about 0.001% to 0.5%; Linatural MBS-I, natural preservative (Lincoln Fine Ingredients) in the range of about 0.5 to 4.0%; and Menthol, a skin toner and a topical bacteriostatic complimentary to Linatural, the preservative (Flavor and Fragrance Specialties) in the range of about 0.01% to 2.0%; Fragrance (Flavor and Fragrance Specialties) in the range of about 0.1% to 0.8%; dimethicone, as cetyl dimethicone (BASF, The Chemical Co.) in the range of about 1.0% to 10%; purified water in the sufficient amount to make a working cream, ranging from about 10% to 90% of the preparation. Cetiol Ultimate may be used instead of dimethicone. All these ingredients contribute to the composition of a facial and body cream composed of all natural ingredients. Other ingredients like Mineral Oil (Witco Co.) of any type and different viscosities, but preferably the one identified as “Carnation,” can be used as a partial or total replacement of olive oil.

### Method of Preparation

The colostrum powder is dissolved in water heated at about 60° C. and preferably 50° C. to 90° C. When all dissolved, the preservative is added (Linatural MBS-1) and mixed very well. Separately, the oil soluble ingredients are dispersed at room temperature into the olive oil (lecithin, vitamin E, vitamin A, dimethicone and menthol) to a very liquid state.

To the colostrum-water solution which has now a temperature of about 40° C. to 60° C., add Sepigel and start to agitate with any type and suitable high sheer mixer to form a cream mixture. Slowly add the olive oil blend and allow to disperse and integrate into the water phase. When. very well dispersed add the fragrance and continue to agitate at high sheer. When all agitated and the cream forms a nice creamy peak, the cream is ready for packaging. The cream is than dispensed by the means of a piston filler in pre-measured quantities into selected jars or plastic tubes or any suitable consumer container package.

### EXAMPLES

The following examples indicate the composition of different functional specific creams to be specific for beautification of different part of the body, and are not meant to be limiting:

### Example 1 Facial Wrinkle Cream

12162014-C-1

| | | Batch size |
|---|---|---|
| Formula | % | 350 gr. |
| Water (DI and hot) | 61.630 | 308.15 |
| Colostrum (Bovine & pwd) | 0.500 | 2.50 |
| Sepigel E1-305 | 5.000 | 25.00 |
| Olive oil (acidity 1.5%) | 25.000 | 125.00 |
| Lecithin | 0.300 | 1.50 |
| Vitamin E (700 IU) | 0.500 | 2.50 |
| Vitamin A/Palmitate | 0.010 | 0.05 |
| Linatural MBS-1 (preserv) | 2.000 | 10.00 |
| Menthol (Crystal) | 0.030 | 0.15 |
| Fragrance FFS #55174 | 0.030 | 0.15 |
| Cetyl Dimethicone | 5.000 | 25.00 |
| Total | 100.000 | 500.00 |

### Method of Preparation

Colostrum and preservative are dissolved into a relatively warm purified water to form a very uniform solution. The oil soluble ingredients (including vitamins A and E, lecithin menthol, dimenthecone and fragrance) are dispersed into the olive oil at room temperature, Sepagel, the emulsifier of choice, is added to form a cream under sustained sheer while slowly the oil blend is added to further intensify the creamy texture being formed under high sheer. The resulting cream is then placed into a suitable container or jars as desired at about a temperature range of 40° C. to 60° C. and allowed to cool.

### Example 2 Face and Neck Cream

12162014-C-2

| | | Batch size |
|---|---|---|
| Formula | % | 350 gr. |
| Water (DI and hot) | 65.200 | 326.00 |
| Colostrum (Bovine & pwd) | 0.500 | 2.50 |
| Sepidel E1-305 | 5.000 | 25.00 |
| Olive oil (acidity 1.5%) | 25.000 | 125.00 |
| Lecithin | 0.300 | 1.50 |
| Vitamin E (700 IU) | 2.000 | 10.00 |
| Linatural MBS-1 (preserv.) | 2.000 | 10.00 |
| Total | 100.000 | 500.00 |

### Method of Preparation

Colostrum and preservative are dissolved into a relatively warm purified water to form a very uniform solution. The oil soluble ingredients (including vitamins A and E, lecithin, menthol, dimethicone, and fragrance) are dispersed into the olive oil at room temperature. Sepagel, the emulsifier of choice, is added to form a cream under sustained sheer while slowly the oil blend is added to further intensify the creamy texture being formed under high sheer. The resulting cream is than placed into suitable container or jars as desired at about a temperature range of 40° C. to 60° C. and allowed to cool.

### Example 3 Hand and Body Cream

12162014-A-3

| | | Batch |
|---|---|---|
| | | size 350 |
| Formula | % | gr. |
| Water (DI and hot) | 63.170 | 315.85 |
| Colostrum (Bovine & pwd) | 0.500 | 2.50 |
| Sepigel E1-305 | 5.000 | 25.00 |
| Olive oil (acidity 1.5%) | 25.000 | 125.00 |
| Lecithin | 0.300 | 1.50 |
| Vitamin E (700 IU) | 4.000 | 20.00 |
| Linatural MBS-1 (preserv) | 2.000 | 10.00 |
| Fragrance FFS #55174 | 0.030 | 0.15 |
| Total | 100.000 | 500.00 |

### Method of Preparation

Colostrum and preservative are dissolved into a relatively warm purified water to form a very uniform solution. The oil soluble ingredients (including vitamins A and E, lecithin menthol, dimenthecone and fragrance) are dispersed into the olive oil at room temperature. Sepagel, the emulsifier of choice is added to form a cream under sustained sheer while slowly the oil blend is added to further intensify the creamy texture being formed under high sheer. The resulting cream is than placed into suitable container or jars as desired at about a temperature range of 40° C. to 60° C. and allowed to cool.

### Example 4—Wrinkle Cream (Olive Oil Reduced by 50% and Replaced With Mineral Oil)

05071214 E-1 Complete

| | | Batch |
|---|---|---|
| | | size 500 |
| Formula | % | gr |
| Water | 65.46 | 327.30 |
| Colostrum (powder) | 0.50 | 2.50 |
| Sepigel 305 or (EI 305) | 5.00 | 25.00 |
| Mineral oil (Carnation) | 12.50 | 62.50 |
| Olive oil | 12.50 | 62.50 |
| Lecithin | 0.30 | 1.50 |
| Vitamin E 70,000 IU | 0.50 | 2.50 |
| Vitamin A/Beta Carotene | 0.01 | 0.05 |
| Linatural MBS-1 (preserv) | 2.00 | 10.00 |
| Menthol (crystal) | 0.03 | 0.15 |
| Fragrance FFS (1111-2) | 0.20 | 1.00 |
| Dimethicone | 1.00 | 5.00 |
| Total | 100.00 | 500.00 |

### Method of Preparation

Colostrum and preservative are dissolved into a relatively warm purified water to form a very uniform solution. The oil soluble ingredients (including vitamins A and E, lecithin menthol, dimenthecone and fragrance) are dispersed into the olive oil at room temperature. Sepagel, the emulsifier of choice is added to form a cream under sustained sheer while slowly the oil blend is added to further intensify the creamy texture being formed under high sheer. The resulting cream is than placed into suitable container or jars as desired at about a temperature range of 40° C. to 60° C. and allowed to cool.

### Example 5—Wrinkle Cream (Olive Oil Replaced Entirely by Mineral Oil)

05071214 D-1 Complete

| | | Batch |
|---|---|---|
| | | size 500 |
| Formula | % | gr |
| Water | 65.46 | 327.30 |
| Colostrum (powder) | 0.50 | 2.50 |
| Sepigel 305 or (EI 305) | 5.00 | 25.00 |
| Mineral oil (Carnation) | 25.00 | 125.00 |
| Lecithin | 0.30 | 1.50 |
| Vitamin E 70,000 IU | 0.50 | 2.50 |
| Vitamin A/Beta Carotene | 0.01 | 0.05 |
| Linatural MBS-1 (presev) | 2.00 | 10.00 |
| Menthol (crystal) | 0.03 | 0.15 |
| Fragrance FFS (111-2) | 0.20 | 1.00 |
| Dimethicone | 1.00 | 5.00 |
| Total | 100.00 | 500.00 |

### Method of Preparation

Colostrum and preservative are dissolved into a relatively warm purified water to form a very uniform solution. Sepagel, the emulsifier of choice is added to form a cream under sustained sheer while slowly the oil blend (including oil soluble ingredients) is added to further intensify the creamy texture being formed under high sheer. The resulting cream is than placed into suitable container or jars as needed at about a temperature range of 40° C. to 60° C.

These cream preparations provide a combination of all-natural ingredients for the beautification of the body and the preservation of skin freshness.

1. A skin cream, comprising:
about 0.5 to 15% of colostrum;
about 0.5 to 15% of a polyacrylamide based emulsifier;
about 1.0 to 20% of an emulsifier containing lecithin or a derivative thereof;
about 1.0 to 20% of olive oil, having acidity ranging from 0.2 to 2.0% and a usage range of 5.0% to 50%;
1.0 to 10% of dimethicone;
0.01% to 4.0% of alpha-tocopherol or an active derivative thereof;
0.01% to 0.5% of vitamin A or an active vitamin A derivative;
0.5 to 4.0% of a natural preservative; and
10 to 90% of purified water to make up 100 percent.

2. A skin cream as set forth in claim 1 wherein the colostrum is bovine, ovine, porcine, seuine, or human derived, or a mixture thereof.

3. A skin cream as set forth in claim 1, wherein the dimethicone is cetyl dimethicone.

4. A skin cream as set forth in claim 1, wherein about half of the olive oil is replaced by mineral oil.

5. A skin cream as set forth in claim 4, where all of the olive oil is replaced by mineral oil.

6. The skin cream according to claim 1, wherein the skin cream lessens the appearance of wrinkles.

7. The skin cream according to claim 1, wherein the skin cream makes the skin appear smoother.

8. A method of improving skin wrinkles comprising applying an effective amount of a skin cream composition according to claim 1.

9. A method of improving skin wrinkles comprising applying an effective amount of a skin cream composition according to claim 2.

10. A method of improving skin wrinkles comprising applying an effective amount of a skin cream composition according to claim 3.
